(19) United States
(12) Patent Application Publication
(10) Pub. No.: US 2024/0008781 A1
(43) Pub. Date: Jan. 11, 2024

(54) Continuous Glucose Monitor For Hospital Use

(71) Applicant: Stacye Klenke, Columbia, MO (US)

(72) Inventor: Stacye Klenke, Columbia, MO (US)

(21) Appl. No.: 17/859,531

(22) Filed: Jul. 7, 2022

(51) Int. Cl. A61B 5/15; A61B 5/145; A61B 5/1495

(52) U.S. Cl. CPC A61B 5/150969; A61B 5/14532; A61B 5/150427; A61B 5/150396; A61B 5/14535; A61B 5/14546; A61B 5/1495; A61B 5/150854

(57) ABSTRACT

Real time glucose monitoring is the only product of its kind that has the capability of adapting to an ICU's already existing blood monitoring system to communicate real-time blood sugar readings to an ICU monitor. This unprecedented device is uniquely designed with durable, high-quality materials to ensure long-term sustainability and employs advanced Bluetooth technology that works in complement with a patch that manages insulin drips with real time glucose levels.

### BACKGROUND

When a type one DKA patient comes to the ICU, nurses are required to perform blood sugar checks, at least hourly, to support in medical care. This is a time-consuming situation on a typical day and has become even more burdensome during Covid considering nurses have to get fully dressed in isolation gear each time they enter a patient's room to perform the task. There have been no products available as original equipment or as an aftermarket to address this problem.

An apparatus to simplify the process and assist nurses in having to check a patient's blood sugar levels hourly making sure the proper medical care is being given, is not being met by any known device or system at present. There have been no products available as original equipment or as an aftermarket to address this problem either.

### SUMMARY OF THE INVENTION

The main purpose of continuous glucose monitor for hospital use is to provide users with an avant-garde type 1 diabetes patch that will communicate real-time blood sugar readings directly to hospital ICU monitor.

A glucose monitor for hospital use comprising a patch comprising an adhesive on a side thereof and configured to define a hole in a center thereof. The disclosure also comprises a needle for capillary blood sampling comprising a penetrating end and a blunt end received in the center of the patch in an angular relation thereto. The disclosure additionally comprises an electronics module detachably connected to the patch and electronically connected to the needle to monitor blood in the needle. The disclosure further includes a blood test sensor disposed in the electronics module adjacent the needle and configured to communicate a glucose level in the blood to the electronics module.

### BRIEF DESCRIPTION OF THE DRAWINGS

FIG. 1 is a front perspective view of the vital readings from the continuous glucose monitor for hospital use on the hospital monitor in accordance with an embodiment of the present disclosure.

FIG. 2 is a top elevational view of the control for the continuous glucose monitor for hospital use in accordance with an embodiment of the present disclosure.

FIG. 3 is a top elevational view of the continuous glucose monitor for hospital use in accordance with an embodiment of the present disclosure.

FIG. 4 is a side elevational view of the continuous glucose monitor for hospital use in accordance with an embodiment of the present disclosure

Throughout the description, similar reference numbers may be used to identify similar elements depicted in multiple embodiments. Although specific embodiments of the invention have been described and illustrated, the invention is not to be limited to the specific forms or arrangements of parts so described and illustrated. The scope of the invention is to be defined by the claims appended hereto and their equivalents.

### DETAILED DESCRIPTION

Reference will now be made to exemplary embodiments illustrated in the drawings and specific language will be used herein to describe the same. It will nevertheless be understood that no limitation of the scope of the disclosure is thereby intended. Alterations and further modifications of the inventive features illustrated herein and additional applications of the principles of the inventions as illustrated herein, which would occur to one skilled in the relevant art and having possession of this disclosure, are to be considered within the scope of the invention.

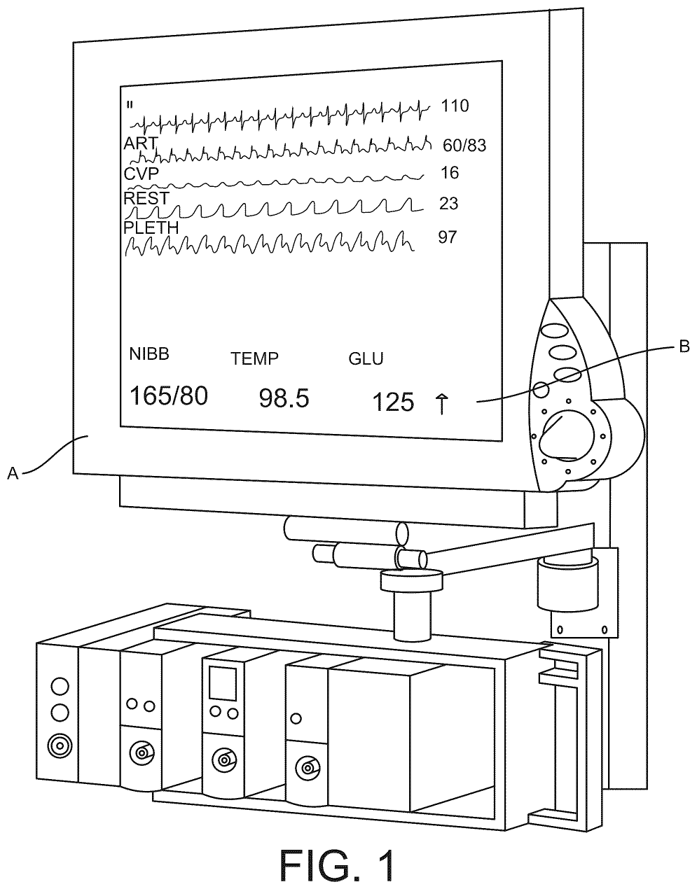

FIG. 1 is a front perspective view of the vital readings from the continuous glucose monitor for hospital use on the hospital monitor showing: ICU monitor referenced as A, and new component reading referenced as B, in accordance with an embodiment of the present disclosure.

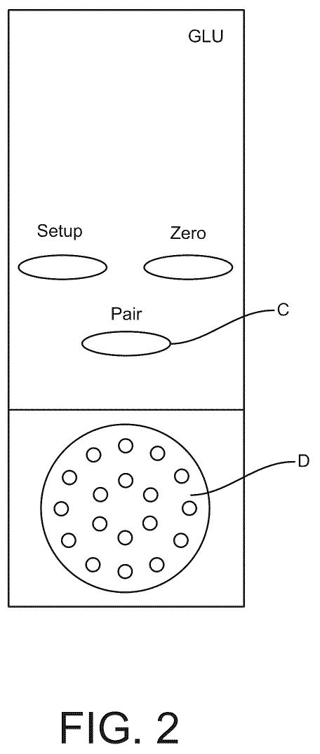

FIG. 2 is a top elevational view of the control for the continuous glucose monitor for hospital use showing: Bluetooth connectivity referenced as C, and not needed with Bluetooth referenced as D, in accordance with an embodiment of the present disclosure.

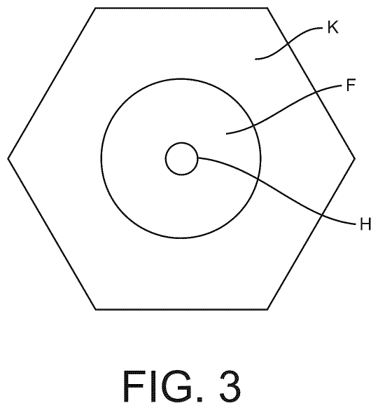

FIG. 3 is a top elevational view of the continuous glucose monitor for hospital use showing: patch top side referenced as K, and needle port referenced as H, and the wireless telemetry module F in accordance with an embodiment of the present disclosure. A perimeter of the patch may be hexagonal as shown or circular or polygonal in embodiments.

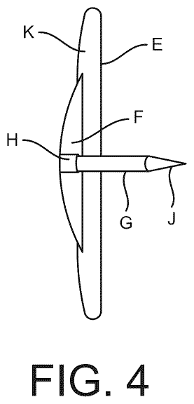

FIG. 4 is a side elevational view of the continuous glucose monitor for hospital use showing: adhesive referenced as E, the electronics module F including wireless telemetry and Bluetooth components & glucose instrumentation and the needle G with a penetrating end J, a needle portal referenced as H, and the patch top side K in accordance with an embodiment of the present disclosure.

The present disclosed continuous glucose monitor for hospital use, also known as “Real Time Glucose Monitoring” offers an improved medium that allows nurses to monitor insulin drips to gauge real-time glucose levels as they are conveyed to a primary monitoring system without having to physically engage the patient, every hour. Real time glucose monitoring introduces a novel patch that is adapted to communicate remotely with ICU's monitor system which permits medical staff to monitor trends of glucose levels quickly and efficiently sans direct contact. Furthermore, at home users are able to wear the patch in order to monitor their real-time blood sugar readings on their phones. Some home users with existing systems with compatibility needs are addressed by this disclosure as well. This approach will not only allow the medical staff to proactively intervene upon concerns of blood results but also enable nurses to monitor readings from a safe, no-contact position, outside a patient's room at a central station.

This innovative, top-quality device enhances the functionality of existing technology by improving the process and overall provides better time management of ICU nurses, reduces distress of the patient, and ensures providers are able to predict outcomes based on blood sugar trends. Real time glucose monitoring may serve to benefit the patient in reducing blood draws, but would further benefit the provider by ensuring current information with respect to blood markers.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

What is claimed is:

1. A glucose monitor for hospital use comprising:
a patch comprising an adhesive on a side thereof and configured to define a hole in a center thereof;
a needle for blood sampling comprising a penetrating end and a blunt end received in the center of the patch in an angular relation thereto;
an electronics module detachably connected to the patch and electroncially connected to the needle to monitor a blood in the needle; and
a blood test sensor disposed in the electronics module adjacent the needle and configured to communicate a glucose level in the blood to the electronics module.

2. The glucose monitor for hospital use of claim 1, further comprising a remote portal configured to calibrate the electronics module.

3. The glucose monitor for hospital use of claim 1, further comprising a remote portal configured to pair the electronics module with the remote portal.

4. The glucose monitor for hospital use of claim 1, further comprising a remote display of the glucose level in the blood telemetrically communicated from the electronics module thereto.

5. The glucose monitor for hospital use of claim 1, wherein the penetrating end of the needle is sharp for skin puncture.

6. The glucose monitor for hospital use of claim 1, wherein the penetrating end of the needle is blunt for cannula use.

7. The glucose monitor for hospital use of claim 1, wherein the blunt end of the needle extends through the patch and is exposed there above for a cannula insertion.

8. The glucose monitor for hospital use of claim 1, wherein the blood test sensor senses a hematocrit in the blood.

9. The glucose monitor for hospital use of claim 1, wherein the blood test sensor further senses an infection in the blood.

10. The glucose monitor for hospital use of claim 1, wherein the blood test sensor further senses a clotting in the blood.

11. The glucose monitor for hospital use of claim 1, wherein the blood test sensor further senses an anemia in the blood.

12. The glucose monitor for hospital use of claim 1, wherein the blood test sensor further senses a protein in the blood.

13. The glucose monitor for hospital use of claim 1, wherein the angular relation of the needle to the patch is a 90 degree fixed relation.

14. The glucose monitor for hospital use of claim 1, wherein the electronics module further comprises circuits for telemetry of data from the blood test sensor to a hospital monitor.

15. The glucose monitor for hospital use of claim 1, wherein the electronics module further comprises circuits for telemetry of data from the blood test sensor to a cell phone.
